(19) United States
(12) Patent Application Publication
(10) Pub. No.: US 2006/0110420 A1
(43) Pub. Date: May 25, 2006

(54) Synthetic pheromone compositions

(75) Inventors: Walter S. Leal, Davis, CA (US); Douglas J. Pesak, Oxford, CT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Regents of the University of California, Oakland, CA (US); Bedoukian Research, Inc, Danbury, CT (US)

(21) Appl. No.: 10/997,503

(22) Filed: Nov. 24, 2004

(51) Int. Cl. A01N 25/00; C07C 9/00

(52) U.S. Cl. 424/405; 554/223; 585/16

(57) ABSTRACT

The present invention provides compounds useful for preparing synthetic pheromone compositions that can be used as attractants or inhibitors of insect species. The compositions are useful in the control of navel orangeworm or meal moth insect pests.

### BACKGROUND OF THE INVENTION

Female-produced sex pheromones in moths (Lepidoptera) are normally complex mixtures of straight chain acetates, aldehydes, and alcohols, with 10-18 carbon atoms and up to three unsaturations. This group of pheromones, Type I according to Ando's classification (Ando et al., Top Curr Chem 239:51-96 (2004)) comprises ca. 75% of the known pheromones. A second major group, Type II (15%) (Ando et al., Top Curr Chem 239:51-96 (2004)) consists of polyunsaturated (up to four double bonds) hydrocarbons and epoxy derivatives with long straight chain (C₁₇-C₂₃) (Ando et al., Top Curr Chem 239:51-96 (2004)). While Type I pheromones are synthesized de novo (Ando et al., Top Curr Chem 239:51-96 (2004); Jurenka, R., Top Curr Chem 239:97-132 (2004)), polyunsaturated hydrocarbons seem to be derived from dietary linoleic and linolenic acid (Jurenka, R., Top Curr Chem 239:97-132 (2004); Ando et al., Top Curr Chem 239:51-96 (2004)).

The major constituent of the sex pheromones of two species in the family Pyralidae, the navel orangeworm, Amyelois transitella Walker (subfamily: Phycitinae) (Coffelt et al., J Chem Ecol 5:955-966 (1979)) and the meal moth, Pyralis farinalis Linnaeus (subfamily: Pyralinae) (Landolt, P. J. and Curtis, C. E., J Kansas Entomol Soc 55:248-252 (1982)) has been previously identified as (Z,Z)-11,13-hexadecadienal belonging to Type I (Ando et al., Top Curr Chem 239:51-96 (2004)). It has been suggested that additional pheromone components may be present in the female navel orangeworm moths (Shorey, H H., Gerber, R. G., Environ Entomol 25:1154-1157 (1996)), but hitherto conventional approaches have failed to identify the full pheromone system. The present invention addresses these and other needs.

### BRIEF SUMMARY OF THE INVENTION

The present invention provides synthetic pheromone compositions useful for attracting, inhibiting or controlling target insect pests. In one embodiment, the present invention provides an isolated compound selected from the group consisting of ethyl-11,13-hexadecadienoate, 3,6,9,12,15-tricosapentaene and 3,6,9,12,15-pentacosapentaene.

In a second embodiment, the present invention provides a synthetic pheromone composition comprising comprising at least one straight-chain pentaene having at least about 19 carbon atoms in the chain. In the typical embodiment, the chain will comprise an odd number of carbon atoms. For example, the synthetic pheromone compositions may comprise (Z,Z,Z,Z,Z)-3,6,9,12,15-tricosapentaene and (Z,Z,Z,Z,Z)-3,6,9,12,15-pentacosapentaene. In certain preferred embodiments, the compositions comprise (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate and ethyl (Z,Z)-11,13-hexadecadienoate. In other embodiments, the compositions comprise (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate, ethyl (Z,Z)-11,13-hexadecadienoate and (Z,Z)-11,13-hexadecadien-1-yl acetate.

In a third embodiment, the present invention provides insect pest traps comprising a trap and a synthetic pheromone composition of the invention.

In a fourth embodiment, the present invention provides methods for attracting an insect pest using an insect pest trap comprising a trap and a synthetic pheromone composition of the invention.

In a fifth embodiment, the present invention provides methods of mating disruption using a synthetic pheromone composition of the invention.

In a fifth embodiment, the present invention provides a method for inhibiting an insect pest using a synthetic pheromone composition of the invention.

### BRIEF DESCRIPTION OF THE DRAWINGS

FIG. 1 (A) Left: Scanning electron micrograph (magnification, 300×) of a male antennae of the navel orange worm. Right: Electrophysiological recording from one of these sensilla trichodea stimulated by 5 female-equivalent of a gland extract. The bar represents the stimulus duration (1 s). GC-EAD recordings from the 3% (B) and hexane (C) fractions after separation of the crude extract by a silica gel column. The peaks highlighted (arrows) in the EAD traces were highly reproducible (N=20). Isomers of the known pheromone (Z,Z)-11,13-hexadecadienal (ALD) generated a cluster of peaks (open arrow).

FIG. 2 MS and vapor-phase IR data of the novel natural products. (A): MS of ethyl (Z,Z)-11,13-hexadecadienoate. (B) MS of (Z,Z)-11,13-hexadecadien-1-yl acetate. (C) MS of (Z,Z,Z,Z,Z)-3,6,9,12,15-tricosapentaene; IR data of the synthetic and natural (inset) compound. (D) MS data of (Z,Z,Z,Z,Z)-3,6,9,12,15-pentacosapentaene.

FIG. 3 Captures of the navel orangeworm and meal moth in traps baited with virgin females of the navel orangeworm and synthetic pheromone mixtures. (A) Catches of male navel orangeworm in traps baited with the previously identified constituent (ALD), full pheromone mixture and virgin female. (B) Catches of the meal moth in Davis, Calif. in traps baited with virgin females of the navel orangeworm and pheromone mixtures. Note that catches of the meal moth in traps baited with virgin females of the navel orangeworm are completely shut off by the addition of 4, (Z,Z)-11,13-hexadecadien-1-yl acetate. Captures in traps loaded with the synthetic mixture devoid of 4 were significantly higher than in traps baited with virgin females of the navel orangeworm, indicating that the natural behavioral antagonist fends off the male meal moth.

### DETAILED DESCRIPTION OF THE INVENTION

The present invention is based on the application of molecular- and sensory physiology-based approaches to the characterization of the full pheromone system in the navel orangeworm, a major pest of almond, pistachio, and walnuts in California. As described below, the sex pheromone system of A. transitella is in fact a hybrid of the two types of pheromones, i.e., a combination of aldehyde, acetate, ethyl ester, and novel highly unsaturated hydrocarbons. Using this information a number of synthetic pheromone compositions for control of insect pests can be prepared.

### I. DEFINITIONS

As used herein, the term “attracting” refers to the action of causing an insect pest, either directly or indirectly, to move in a direction towards the source of stimulus. One of skill in the art will recognize that suitable stimuli include thermostimuli, mechanostimuli, for example, airborne sound waves, or substrate borne pressure waves, electromagnetic stimulus including visual stimulus such as patterns, objects, color, light, and chemical stimulus including pheromones. A chemical stimulus can be an individual compound or a composition, including more than one compound, that either directly or indirectly, causes the insect to move toward the source of the stimulus.

As used herein, the term “inhibiting” refers to the action of causing an insect pest, either directly or indirectly, to not move in a direction towards the source of stimulus. One of skill in the art will recognize that suitable stimuli include thermostimuli, mechanostimuli, for example, airborne sound waves, or substrate borne pressure waves, electromagnetic stimulus including visual stimulus such as patterns, objects, color, light, and chemical stimulus including pheromones. A chemical stimulus can be an individual compound or a composition, including more than one compound, that either directly or indirectly, causes the insect to fail to move in a direction toward the source of the stimulus. Useful stimuli include those that also repel, or drive away, insect pests of the present invention.

As used herein, the term “insect pest” refers to any insect that is disruptive or destructive to the growth and development of agricultural crops. Examples of agricultural crops useful in the present invention include, but are not limited to, almonds, walnuts and pistachios. In some embodiments, insect pests of the present invention belong to the family Pyralidae. In other embodiments, insect pests of the present invention belong to the subfamily Phycitinae or Pyralinae. In still other embodiments, insect pests of the present invention include the navel orangeworm, Amyelois transitella Walker, and the meal moth, Pyralis farinalis Linnaeus. One of skill in the art will recognize that further insect pests will be useful in the present invention.

As used herein, the term “isolated” refers to a substance that has been separated from one or more substances so as to obtain pure or in a free state. In some embodiments, methods of isolation include crystallization and chromatography. Other methods of isolation will be apparent to one of skill in the art.

As used herein, the term “straight-chain” refers to a hydrocarbon molecule that is acyclic and unbranched.

As used herein, the term “synthetic pheromone composition” refers to a chemical composition of one or more specific isolated pheromone compounds. Typically, such compounds are produced synthetically and mimic the response of natural pheromones. Pheromones are compounds produced by an animal or insect and serve as a stimulus to other individuals of the same species for one or more behavioral responses. In some embodiments, the behavioral response to the pheromone is attraction. In other embodiments, the species to be influenced is repelled by the pheromone. In these embodiments, the pheromone is an inhibitor.

As used herein, the term “trap” refers to any device into which the synthetic pheromone compositions of the present invention are placed, and that prevents the insect pest from escaping once the insect pest has come into contact with the trap. The present invention provides traps that can be of various sizes, shapes, colors, and materials. Traps of the present invention can be designed and manufactured specifically for use as an insect trap, or can be a container converted and adapted from other uses such as, for example, a glass Petri dish, a metal coffee can, a cardboard box, or any ordinary plastic, metal, fiberglass, composite or ceramic container. Preferred materials for use in making the traps of the present invention include, but are not limited to, cardboard, metal, metal alloys, glass, paper, plastic, acrylic, fiberglass, composite, and ceramic. The traps of the present invention preferably have a bottom, sidewalls, and a top. The bottom, sidewalls and top of the trap can be solid, or be perforated. An example of a perforated sidewall is a screen. The traps are configured such that insect pests can enter the trap but are unable to escape once inside the trap. Other useful traps of the present invention are commercially available (for example, from Trece Inc.).

As used herein, the term “mating disruption” refers to the release of synthetic pheromone compositions (e.g., using controlled release from polymers comprising the pheromone, or by automated aerosol dispensers) in sufficient quantities that males are unable to orient to natural sources of pheromone, fail to locate females, and reproduction is thus prevented.

### II. COMPOUNDS

The compounds of the present invention are useful for preparing synthetic pheromone compositions that can be used as attractants or inhibitors of insect species. Use of synthetic pheromone compositions for control insect pests is well known in the art. One of skill in the art can conveniently use the compounds of the invention in the preparation of synthetic pheromone compositions useful in a variety of contexts. Exemplary methods for preparing the compounds of the present invention are described in the Examples section below.

In one embodiment, the present invention provides an isolated compound selected from the group consisting of ethyl-11,13-hexadecadienoate, 3,6,9,12,15-tricosapentaene and 3,6,9,12,15-pentacosapentaene. In another embodiment, the present invention provides an isolated compound selected from the group consisting of ethyl (Z,Z)-11,13-hexadecadienoate, (Z,Z,Z,Z,Z)-3,6,9,12,15-tricosapentaene and (Z,Z,Z,Z,Z)-3,6,9,12,15-pentacosapentaene.

### III. SYNTHETIC PHEROMONE COMPOSITIONS

The synthetic pheromone compositions of the present invention are useful for attracting, inhibiting or controlling a number of insect pests. As explained in detail below, the compositions are conveniently used for control of the navel orangeworm and the meal moth. In some embodiments, the synthetic pheromone compositions of the present invention are useful for inhibiting the meal moth.

Synthetic pheromone compositions can be conveniently tested in the assays described below. For example, the synthetic pheromone compositions of the present invention can be tested to determine affinity for a pheromone-binding protein (AtraPBP) present in the navel orangeworm. Alternatively, the compositions can be tested for the ability to stimulate the olfactory receptor neurons (ORNs) in the insect's sensilla trichodea producing a response that indicates the presence or absence of a pheromone. In a typical embodiment, the compositions stimulate an electroantennogram response from an insect pest antenna, as described below.

A synthetic pheromone composition of the invention may comprise one or more of the isolated compounds disclosed here. For example, a minimal synthetic pheromone composition may comprise 3,6,9,12,15-tricosapentaene or 3,6,9,12,15-pentacosapentaene. In a preferred embodiment, the synthetic pheromone composition comprises (Z,Z,Z,Z,Z)-3,6,9,12,15-tricosapentaene and (Z,Z,Z,Z,Z)-3,6,9,12,15-pentacosapentaene.

In a typical embodiment, the present invention provides a synthetic pheromone composition comprising 3,6,9,12,15-tricosapentaene, 3,6,9,12,15-pentacosapentaene, 11,13-hexadecadienal, ethyl palmitate and ethyl-11,13-hexadecadienoate. In a preferred embodiment, the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate and ethyl (Z,Z)-11,13-hexadecadienoate. Such synthetic pheromone compositions are useful, for example, in attracting or controlling the navel orangeworm and meal moth.

In some embodiments, the present invention provides a synthetic pheromone composition comprising 3,6,9,12,15-tricosapentaene, 3,6,9,12,15-pentacosapentaene, 11,13-hexadecadienal, ethyl palmitate, ethyl-11,13-hexadecadienoate and 11,13-hexadecadien-1-yl acetate. In preferred embodiments, the present invention provides a synthetic pheromone composition comprising (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate, ethyl (Z,Z)-11,13-hexadecadienoate and (Z,Z)-11,13-hexadecadien-1-yl acetate. Such synthetic pheromone compositions are useful, for example, in attracting the navel orangeworm and repel the meal moth. These compositions contain an antagonist of the meal moth, which operates to inhibit the meal moth.

The particular ratio of the compounds in the synthetic pheromone compositions of the invention is not a critical aspect of the invention. For example, the present invention provides a synthetic pheromone composition comprising compounds in about the following ratio: 3,6,9,12,15-tricosapentaene, 1-40; 3,6,9,12,15-pentacosapentaene, 1-50; 11,13-hexadecadienal, 100; ethyl palmitate, 0-15; ethyl 11,13-hexadecadienoate, 0-10; and 11,13-hexadecadien-1-yl acetate, 0-10. A preferred composition comprises the compounds in about the following ratio: 3,6,9,12,15-tricosapentaene, 15; 3,6,9,12,1 5-pentacosapentaene, 17; 11,13-hexadecadienal, 100; ethyl palmitate, 14; ethyl 11,13-hexadecadienoate, 5; and 11,13-hexadecadien-1-yl acetate, 5. One of skill in the art will recognize that other similar ratios of compounds for the synthetic pheromone compositions of the present invention are also useful.

### IV. INSECT PEST TRAPS

The present invention also provides an insect pest trap comprising a synthetic pheromone composition of the invention. The compositions typically comprise at least one straight-chain pentaene having at least about 19 carbon atoms in the chain. In the typical embodiment, the chain will comprise an odd number of carbon atoms. In one embodiment, the present invention provides an insect pest trap wherein the synthetic pheromone composition comprises 3,6,9,12,15-tricosapentaene and 3,6,9,12,15-pentacosapentaene. In another embodiment, the present invention provides an insect pest trap wherein the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate and ethyl (Z,Z)-11,13-hexadecadienoate. In still another embodiment, the present invention provides an insect pest trap wherein the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate, ethyl (Z,Z)-11,13-hexadecadienoate and (Z,Z)-11,13-hexadecadien-1-yl acetate.

In other embodiments, the synthetic pheromone composition of the present invention is formulated in rubber septa or in disks. One of skill in the art will recognize that other formulations are useful in the present invention.

### V. METHODS FOR ATTRACTING AN INSECT PEST

The present invention further provides a method for attracting an insect pest using an insect pest trap comprising a trap and a synthetic pheromone composition comprising 3,6,9,12,15-tricosapentaene and 3,6,9,12,15-pentacosapentaene. In one embodiment, the method for attracting an insect pest comprises a synthetic pheromone composition comprising (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate and ethyl (Z,Z)-11,13-hexadecadienoate. In another embodiment, the method for attracting an insect pest comprises a synthetic pheromone composition comprising (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate, ethyl (Z,Z)-11,13-hexadecadienoate and (Z,Z)-11,13-hexadecadien-1-yl acetate.

One of skill will recognize that the manner in which the pest traps of the invention are used will depend upon the particular pest to be controlled or crop to be protected. In some embodiments, the insect pest is from the species family Pyralidae. In preferred embodiments, the method is used for attracting or repelling an insect pest from the subfamily of Phycitinae or Pyralinae.

In a typical embodiment, the present invention provides a method for controlling an insect pest from the subfamily Phycitinae. In another embodiment, the present invention provides a method for attracting an insect pest such as the navel orangeworm, Amyelois transitella Walker.

In still other embodiments, the present invention provides a method for attracting an insect pest from the subfamily Pyralinae. In another embodiment, the present invention provides a method for attracting an insect pest such as the meal moth, Pyralis farinalis Linnaeus.

### VI. METHODS OF DISRUPTING MATING

Use of synthetic pheromone compositions to disrupt mating of insect pests is well known in the art. Release of high and uniform concentrations of the pheromone are thought to shut down the ability of male sensory organs to detect the pheromone. In addition, if the pheromones are released from many sources males are attracted to false sources, wasting time and energy. Under these conditions, the likelihood of a male finding a female is reduced.

A number of devices that provide a synthetic pheromone reservoir and controlled release of the contents are known. For example, a common method relies upon evaporation from polymers impregnated or filled with pheromone. Such devices are typically composed of rubber and plastic in sizes ranging from sprayed microcapsules to long strips hung on trees. Such devices can be open-ended hollow fibers or hollow tubes having their lumen filled with the composition and sealed at the end. In addition, automatated aerosol dispensers can be used.

### VII. EXAMPLES

General

Gas chromatography-mass spectrometry (GC-MS) was obtained with a 5973 Network Mass Selective Detector linked to a 6890 Network GC System (Agilent Technologies, Palo Alto, Calif.) operated either in the electron impact (EI) or chemical ionization (CI) mode. Chromatographic resolution was done on an HP-5MS column (30 m×0.25 mm; 0.25 μm; Agilent) that was operated at 70° C. for 1 min, increased to 250° C. at a rate of 10° C./min and held at this temperature for 10 min. Vapor phase infrared spectroscopy was carried out on a Win GC/IR Pro (Varian Inc., formerly Digilab, Randolph, Mass.) with a GC/IR interface and a Scimitar FTS 2000 linked to a 6890 Network GC System (Agilent). Separation was done on a HP-5 column (30 m×0.32 mm; 0.25 μm; Agilent) operated at 100° C. for 1 min, increased to 250° C. at a rate of 20° C./min and held at this temperature for 5 min; the transfer line and light pipe were operated at 250° C. Gas chromatography with electroantennographic detection (GC-EAD) was done with two different systems: HP 5890 and HP 6890 (Agilent) both having Syntech's GC-EAD transfer lines and temperature control units (Hilversum, The Netherlands). In both systems, the effluent from the capillary column was split into EAD and flame ionization detector (FID) in 3:1 ratios. Male antennae were placed in EAG probes (Syntech) and held in place with Spectra 360 electrode gel (Parker Laboratories, Orange, N.J.). These probes were connected to AM-01 amplifiers (Syntech). The analog signals were fed into A/D 35900E interfaces (Agilent) and acquired simultaneously with FID signal on an Agilent Chemstation. Chromatographic separations were done either with HP-5MS column operated as in GC-MS or with HP-INNOWAX column (30 m×0.32 mm; 0.25 μm; Agilent) operated at 70° C. for 1 min, increased to 250° C. at a rate of 10° C./min and held at this temperature for 5 min.

### Example 1

### Identification of Natural Pheromone Components

Insect Rearing, Pheromone Extraction and Fractionation.

The navel orangeworm colony started from larvae collected in Bakersfield, Calif. The larvae were kept in dried and roasted pistachio at 25±2° C., 75±10% relative humidity, and a 16:8 (L:D) photoregime. Adults were transferred to aluminum cages (30×30×30 cm) and kept for 48 h to allow copulation. After the first generation, 20% of the emerged adults were used to maintain the colony. The remainder of the pupae were kept individually in culture tubes (17 mm i.d; 10 cm long). Upon emergence males were used for EAD and SSR and females for gland extracts or trap baits. Pheromone glands of 1- to 2-day-old virgin females were extracted 2 h before photophase for 10 min in glass-distilled hexane and kept at −80° C. until used. Crude extracts were subjected to flash column chromatography on silica gel (60-200 Mesh, Fisher Scientific) by successive elution with hexane-ether mixtures in the following order: 100:0 (hexane fraction), 99:1 (1% fraction), 98:2, 97:3, 95:5, 90:10, 50:50, 0:100.

Single Sensillum Recordings (SSR)

Male moths were immobilized with dental wax on the recording stage of a single sensillum recording unit (Syntech, INR-02), the tip of the sensilla were cut (Kaissling, K.-E. Single unit and electroantennogram recordings-in insect olfactory organs, In: Spielman AI, Brand J G (ed) Experimental Cell Biology of Taste and Olfaction: Current Techniques and Protocols, CRC Press, Boca Raton, pp. 361-386 (1995)) and placed under a stereomicroscope (SZX12, Olympus, Tokyo, Japan). The indifferent (ground) electrode was a thin tungsten electrode inserted into the head. The recording glass electrode was slipped over the cut tip of the sensilla with a Piezo Manipulator (PM-10, World Precision Instruments, Sarasota, Fla.) while the signal was monitored with a Tektronix oscilloscope (TDS-2014). The pre-amplified signal was acquired with an acquisition system (IDAC-USB, Syntech) and SSR software (Autospike 2000, Syntech). The antennal preparation was continuously flushed with clean air at 0.5 m/s. Each stimulus was applied to a filter paper, dried at least 10 min, and placed within a glass cartridge (7 mm i.d.; 5 cm long). The cartridge opening was placed 1 cm in front of the antennae. The stimulus air was delivered by a stimulus controller (CD-02/E, Syntech).

Results

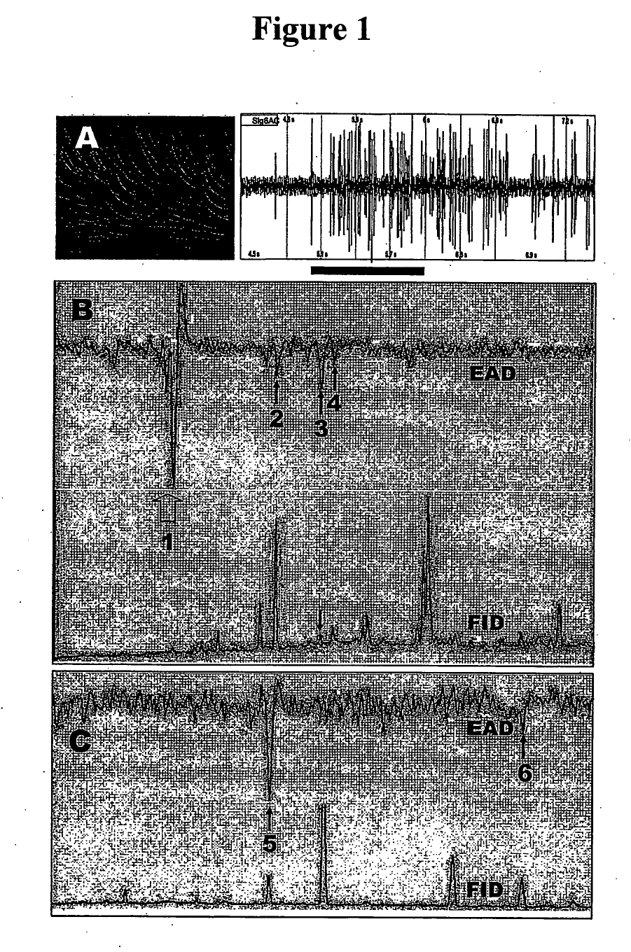

We have taken a comprehensive approach in studying chemical communication in the navel orangeworm, A. transitella. On the one hand, we have isolated, cloned, and expressed pheromone- and odorant-binding proteins. Binding assays with recombinant olfactory proteins indicated that the previously identified pheromone, (Z,Z)-11,13-hexadecadienal (ALD), bound to the major pheromone-binding protein (AtraPBP) with apparent high affinity. Preliminary screening of potential ligands showed that a related acetate compound, (Z,Z)-11,13-hexadecadienyl acetate, had similar affinity to AtraPBP. In addition, electrophysiological recordings from sensilla trichodea (single sensillum recordings, SSR) in male moth antennae indicated that the navel orangeworm possess multiple olfactory receptors neurons (ORN), which are stimulated by constituents in hexane extracts from pheromone glands (FIG. 1A).

The crude extract was fractionated by flash chromatography with electrophysiological activity being monitored by SSR. Different ORNs were stimulated not only by the ALD-containing fractions (5 and 10% ether), but also by two other fractions: hexane (0% ether) and 3% ether. Based on the spike amplitudes, it was not possible to conclude unambiguously whether different ORNs fired or if the SSR responses were derived only from minute amounts of ALD, particularly in the 3% fraction.

To determine the active constituents in these SSR-active fractions (3% and hexane), we used gas chromatography coupled with an electroantennographic detector (GC-EAD) and having male moth antennae as the sensing element. GC-EAD analyses using a non-polar column (HP-5MS) indicated that in addition to the ALD pheromone (peak 1), the 3% fraction contained three other EAD-active peaks (2, 3, and 4) (FIG. 1B), whereas the hexane fraction contained two other EAD-active peaks (5 and 6) (FIG. 1C). The peaks were numbered in the order of their retention times (t_{R}) in a non-polar column (1: t_{R}, 17.30 min; 2: 18.44 min; 3: 18.96 min; 4: 19.08 min; 5: 20.9 min; 6: 23.8 min). The retention times of these EAD-active peaks in a polar column (HP-INNOWAX) were: 16.59, 17.37, 18.32, and 18.72 min (3% fraction) and 18.52 and 20.22 min (hexane fraction). GC-MS analyses indicated that the cluster of peaks (labeled peak 1 in FIG. 1B) is derived from the isomers of the previously identified pheromone, ALD.

Authentic synthetic standards showed the following order of elution by GC-MS: (Z,E)-, (E,Z)-, (Z,Z)-, and (E,E)-1 (t_{R}, 14.77, 14.86, 14.94, and 14.98 min, respectively). The strongest EAD-active peak in the cluster (1) corresponds to the (Z,Z)-isomer, whereas the earlier eluting, small EAD-active peaks are generated by (Z,E)- and (E,Z)-isomers. While the occurrence in gland extracts of the major, (Z,Z)-, and other two minor isomers, i.e., (Z,E) and (E,Z), were clearly observed by both GC-EAD and GC-MS, the (E,E)-isomer was not detectable by these techniques. In SSR experiments, large spike amplitude cells (FIG. 1A) were activated by (Z,Z)-1, whereas synthetic (E,E)-1 activated mainly a small spike ORN, with small activation of a large spike cell.

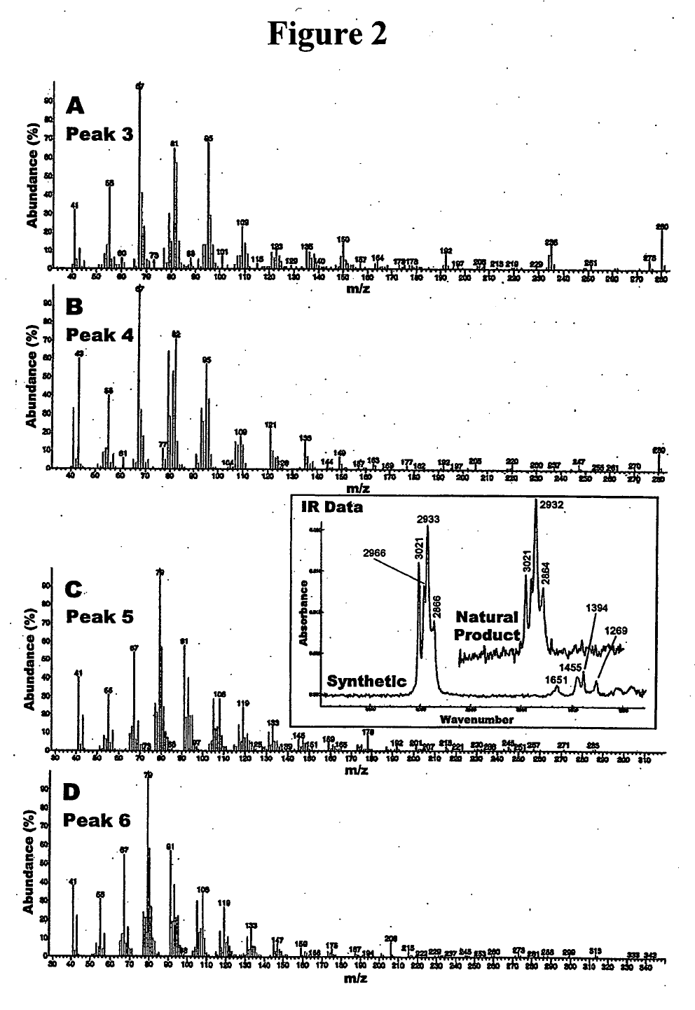

Peak 2 was identified as ethyl palmitate by GC-MS and library (Wiley) search. Co-elution with authentic ethyl palmitate (Aldrich) in polar and non-polar columns and EAD activity confirmed the identification. The fragmentation pattern in the MS of peak 3 (FIG. 2A) somewhat resembles that of the ALD constituent. The loss of 45 (molecular ion peak, m/z 280 and m/z 235) and the peak at m/z 88 suggested that 3 was a di-unsaturated ethyl ester. This assignment was also supported by the vapor phase infrared spectra with a strong carbonyl stretching band at 1753 cm⁻¹, as commonly observed in methyl and ethyl esters (Leal, W. S., Infrared and ultraviolet spectroscopy techniques; In: Millar J G, Haynes K F (ed) Methods in Chemical Ecology: Chemical Methods, Kluwer Academic Publishers, Norwell, pp. 185-206 (1998)). Although it was not possible to assign the location of the double bonds, we suggested on the basis of the MS profile that it might be derived from the same biosynthetic pathway as ALD and, therefore, having the double bonds in positions 11 and 13. Synthetic ethyl (Z,Z)-11,13-hexadecadienoate was indistinguishable from 3 in the MS and GC-IR profiles, retention times in polar and non-polar columns; synthetic 3 was also EAD active.

Peak 4 gave a MS (FIG. 2B) identical to that of synthetic (Z,Z)-11,13-hexadecadien-1-yl acetate, utilized in molecular-based approach for screening of potential attractants (see above). Synthetic and natural compounds have identical retention times in polar and non-polar columns. Synthetic (Z,Z)-11,13-hexadecadien-1-yl acetate showed the same electrophysiological activity as the natural product. In summary the 3% fraction contained four EAD-active peaks, which were fully characterized as 1: (Z,Z)-11,13-hexadecadienal (CAS # 71317-73-2); 2: ethyl palmitate (CAS # 628-97-7); 3: ethyl (Z,Z)-11,13-hexadecadienoate, and 4: (Z,Z)-11,13-hexadecadien-1-yl acetate (CAS # 118744-50-6). Whereas mixtures of biosynthetically related aldehydes and acetates are commonly utilized in moth sex pheromones, this is the first identification of a novel ethyl ester likely derived from the same biosynthetic pathway as that of the major pheromone constituent (ALD).

MS data suggested that 5 and 6 were related compounds (FIG. 2 C,D). The base peak in the MS of 5 (FIG. 2C) appeared at m/z 79; chemical ionization (CI, methane) MS indicated that a tiny peak at m/z 314 was the molecular peak. CI gave two major peaks at m/z 313 ([M−H]⁺) and 315 (base peak, [M+H]⁺). Hydrogenation of the purified compound and MS analyses suggest that 5 is a pentaunsaturated straight chain hydrocarbon. The peak at m/z 178 [Me(CH₂)₆(CH═CH)₃H]⁺ suggest the occurrence of 6 methylenes after the last double bond (Karunen, P., Phytochemistry 13:2209-2213 (1974); Youngblood et al., Marine Biol 8:190-201 (1971); Lee et al., Biochim Biophys Acta 202:386-388 (1970); Blumer et al., Marine Biol 6:226-235 (1970)). The occurrence of a double bond in position 3 was inferred by the fragment [MeCH₂(CH═CH)₃H]⁺ at m/z 108 (Karunen, P., Phytochemistry 13:2209-2213 (1974); Youngblood et al., Marine Biol 8:190-201 (1971); Lee et al., Biochim Biophys Acta 202:386-388 (1970); Blumer et al., Marine Biol 6:226-235 (1970)) and the lack of vinyl CH₂ in vapor phase IR (Leal, W. S., Infrared and ultraviolet spectroscopy techniques; In: Millar J G, Haynes K F (ed) Methods in Chemical Ecology: Chemical Methods, Kluwer Academic Publishers, Norwell, pp. 185-206 (1998)) at ca. 3080 cm⁻¹ (FIG. 2C, inset). IR and MS suggest that there was no conjugation and the strong IR band at 3021 cm⁻¹ suggests that all double bonds had the cis configuration (Leal, W. S., Infrared and ultraviolet spectroscopy techniques; In: Millar J G, Haynes K F (ed) Methods in Chemical Ecology: Chemical Methods, Kluwer Academic Publishers, Norwell, pp. 185-206 (1998)) (FIG. 2C). MS of 6 showed evidence for 8 methylenes after the last double bond: m/z 206, [Me(CH₂)₈(CH═CH)₃H]⁺. (Karunen, P., Phytochemistry 13:2209-2213 (1974); Youngblood et al., Marine Biol 8:190-201 (1971); Lee et al., Biochim Biophys Acta 202:386-388 (1970); Blumer et al., Marine Biol 6:226-235 (1970)) The molecular peak at m/z 342 was confirmed by CI. Like 5, compound 6 showed no band corresponding to vinyl CH₂ in vapor phase IR, no conjugation, and evidence for all-cis configuration. Thus, the two compounds were tentatively identified as (Z,Z,Z,Z,Z)-3,6,9,12,15-tricosapentaene and (Z,Z,Z,Z,Z)-3,6,9,12,15-pentacosapentaene, respectively. The synthetic polyunsaturated hydrocarbons were indistinguishable from the natural products in their MS, IR, and retention times under GC-EAD and GC-MS separation conditions. Even with a shallow separation method in a polar column (INNOWAX; 70° C. to 250° C. at 5° C./min), both synthetic and natural products gave the same retention time: (Z,Z,Z,Z,Z)-3,6,9,12,15-tricosapentaene, 31.33 min; (Z,Z,Z,Z,Z)-3,6,9,12,15-pentacosapentaene, 34.42 min.

The synthetic polyunsaturated hydrocarbons were also EAD-active. Hitherto monoene, diene, triene and tetraene hydrocarbons (C₁₇-C₂₃) have been identified as sex pheromones (Ando et al., Top Curr Chem 239:51-96 (2004)), but pentaenes are not known. Both 5 and 6 are novel types of natural products, but a shorter pentaene, (Z,Z,Z,Z,Z)-3,6,9,12,15-heneicosapentaene (CAS 66887-59-0), has been previously identified from marine benthic algae (Youngblood et al., Marine Biol 8:190-201 (1971)) and spores of a moss (Karunen, P., Phytochemistry 13:2209-2213 (1974)). Given the methylene-interrupted pattern of the 3,6,9 moiety, it is conceivable that these novel moth pheromones (5 and 6) could be derived from linolenic acid after chain elongation, desaturation and decarboxylation, provided the insect possesses the appropriate enzymes.

### Example 2

### (Z,E)-, (E,Z)-, (E,E)-, AND (Z,Z)-11,13-HEXADECADIENAL (1)

The (Z,Z) isomer can be prepared by a previously published method (Sonnet, P. E. and Heath, R. R., J Chem Ecol 6:221-228 (1980). The (Z,E) isomer was prepared by a the sequence shown in Scheme 1-1. (E)-12-pentadecen-10-yn-1-ol THP was prepared by palladium catalyzed cross coupling of 10-undecyn-1-ol THP (prepared from 10-undecyn-1-ol and dihydropyran) with E-1-iodo-1-butene (Zweifel, G. and Whitney, C. C., J Am Chem Soc 89:2753-2754 (1967); Alami et al., Tetrahedron Lett 34:6403-6406 (1993)). Addition of dicyclohexyl borane across the triple bond followed by hydrolysis of both the borane and THP protecting group gave the desired (Z,E) diene stereochemistry (Brown, H. C., Organic Synthesis via Boranes, John Wiley and Sons, New York (1975)). The alcohol was converted to bromide via the mesylate using conventional methods (Jones, R. A., Quaternary ammonium salts, Academic Press.San Diego (2001)). The Grignard reagent of the bromide was then prepared and reacted with triethylorthoformate to give (Z,E)-11,13-Hexadecadienal diethyl acetal (DeWolfe, H. R., Carboxylic ortho acid derivatives, Academic Press.New York (1970)). Acidic hydrolysis (Greene T. W. and Wuts, P. G. M., Protective groups in organic synthesis, John Wiley & Sons.New York (1999)) gave the desired aldehyde. The (E,Z) isomer was prepared by the sequence shown in Scheme 1-2. (E)-10-pentadecen-12-yn-1-ol THP was prepared from the borane adduct of 10-undecyn-1-ol THP and the lithium salt of 1-butyne (Svirskaya et al., J Chem Ecol 10:795-807 (1984)). The rest of the synthesis follows that of the (Z,E) isomer from the THP stage described above. The (E,E) isomer was prepared by isomerization of the (Z,Z) isomer mediated by thiophenol and a radical source (Schwarz et al., J Org Chem 51:260-263 (1986)) followed by fractional crystallization.

### Example 3

### ETHYL (Z,Z)-11,13-HEXADECADIENOATE (3)

(Z,Z)-10,12-Pentadecadien-1-ol can be prepared using the appropriate starting materials using a previously reported reaction sequence (Sonnet, P. E., Heath, R. R., J Chem Ecol 6:221-228 (1980)). The alcohol was converted to bromide (Scheme 1-3). The Grignard reagent of the bromide was prepared and quenched with excess diethylcarbonate (Whitmore F. C. and Loder, D. J., Ethyl, Naphthoate, In: Blott A H (ed) Organic Syntheses; John Wiley & Sons, New York, pp. 282-283 (1943)) to give the desired ester 3.

### Example 4

### (Z,Z)-11,13-HEXADECADIEN-1-YL ACETATE (4)

Compound 4 was prepared by LAH reduction of the aldehyde (Z,Z)-1 followed by acylation of the alcohol with acetyl chloride (Scheme 1-4).

### Example 5

### (Z,Z,Z,Z,Z)-3,6,9,12,15-TRICOSAPENTAENE (5) AND (Z,Z,Z,Z,Z)-3,6,9,12,15-PENTACOSAPENTAENE (6)

Commercially available methyl (Z,Z,Z,Z,Z)-5,8,11,14,17-eicosapentaenoate was reduced to the corresponding alcohol with Red-Al (Málek, J., Reduction by metal alkoxyaluminum hydrides, Part II, Carboxylic acids and derivatives, nitrogen compounds and sulfur compounds; In: Overman, L. (ed) Organic Reactions, John Wiley & Sons, New York, pp. 249 (1988)) (Scheme 1-5). The alcohol was then converted to bromide, which was coupled to either n-propyl or n-pentyl Grignard catalyzed by copper salts (Erdik, E., Tetrahedron Lett 40:641-657 (1984)) to give the 5 and 6 pentaenes, respectively.

### Example 6

### Field Experiments

Experimental

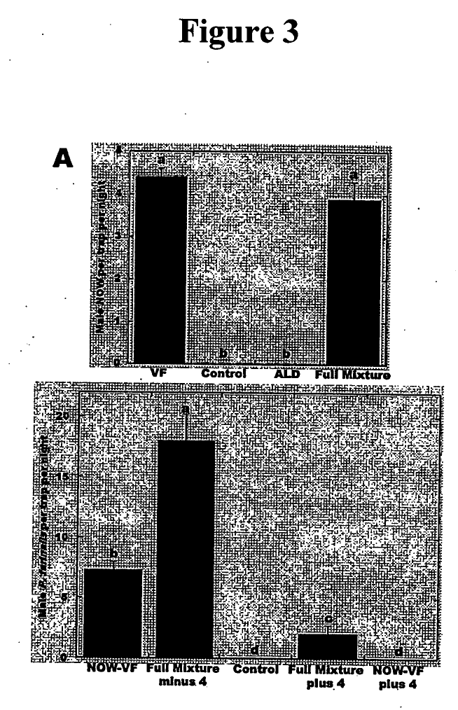

Tests were conducted in almond and walnut plot fields in the UC Davis campus. Pheromone samples (0.5 mg) were formulated in rubber septa or in 12 mm diameter, 3 mm thick discs (made of ES fiber, Chisso Co. Ltd, Tokyo, Japan) and loaded into Pherocon IC traps (Trece Inc., Salinas, Calif.). Three or five 1 to 3-day old virgin females were placed in fiberglass screen cages (Curtis, C. E. and Clark, J. D., J Econ Entomol 77:1057-1061 (1984) Curtis et al., J Econ Entomol 78:1425-1430 (1985)). Baited and control traps were placed at ca. 1.8 m height in randomized blocks with the intertrap distance of ca. 10 m. Capture data were transformed to log (x+0.5) and analyzed by ANOVA. In FIG. 3, treatments followed by the same letters are not significantly different at the 5% level in the Tukey-Kramer honestly significant difference. Means of captures are untransformed, and error bars show one standard error (SE).

Results

The ratio of the six constituents of the sex pheromone system of the navel orangeworm, analyzed by GC with three replicates of gland extracts, was (Z,Z)-11,13-hexadecadienal 100 (850±97 pg/female); ethyl palmitate, 14±1.3; ethyl (Z,Z)-11,13-hexadecadienoate, 4.8±1.4; (Z,Z)-11,13-hexadecadien-1-yl acetate, 4.9±1.2; (Z,Z,Z,Z,Z)-3,6,9,12,15-tricosapentaene, 14.9±2.4; and (Z,Z,Z,Z,Z)-3,6,9,12,15-pentacosapentaene, 17.1±4.3. Preliminary field tests in Davis showed that captures in traps baited with the full mixture of the pheromone system (0.5 mg) did not differ significantly from catches in traps baited with 1- to 3-day-old virgin females (FIG. 3A), whereas traps baited with the single pheromone constituent and control traps captured no moths in 3-wk period of tests.

In some locations, traps baited with virgin females of the navel orangeworm captured also males of the meal moth, P. farinalis. Interestingly, catches of the meal moth were significantly smaller when traps were baited with synthetic sample containing the full pheromone system. Tests with partial mixtures showed that removal of (Z,Z)-11,13-hexadecadien-1-yl acetate increased dramatically captures of male meal moth (FIG. 3B). This compound is a behavioral antagonist, which is not strong enough in the natural pheromone to completely repel the meal moth. This is supported by the complete lack of captures in traps baited with virgin females and boosted with a synthetic sample (0.5 mg/per device) of the acetate. In addition, GC-EAD experiments utilizing antennae of male meal moth captured in the pheromone traps confirmed that P. farinalis male do possess detectors tuned to (Z,Z)-11,13-hexadecadien-1-yl acetate.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

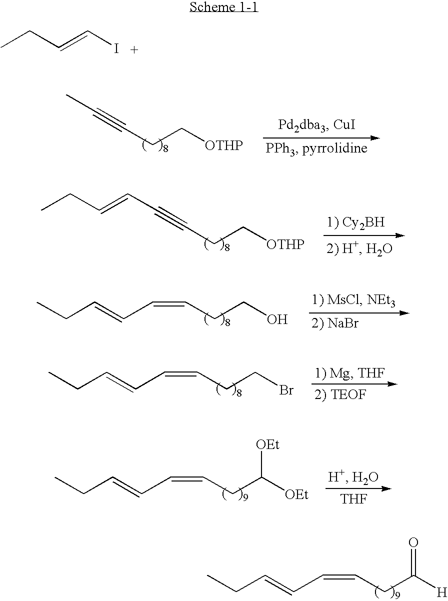

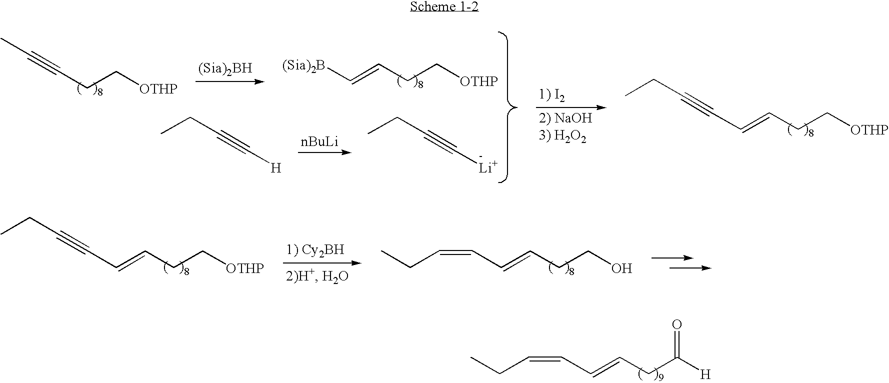

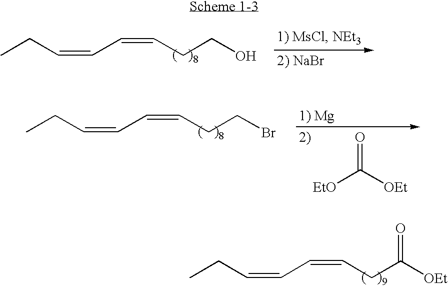

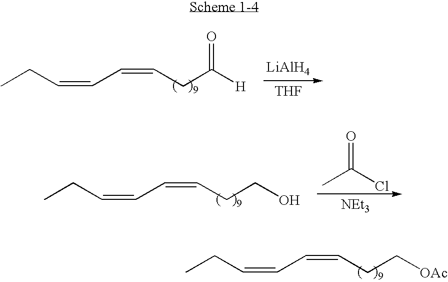

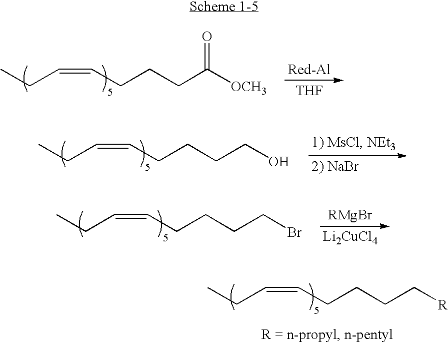

What is claimed is:

1. An isolated compound selected from the group consisting of: ethyl-11,13-hexadecadienoate, 3,6,9,12,15-tricosapentaene and 3,6,9,12,15-pentacosapentaene.

2. The isolated compound of claim 1 selected from the group consisting of: ethyl (Z,Z)-11,13-hexadecadienoate, (Z,Z,Z,Z,Z)-3,6,9,12,15-tricosapentaene and (Z,Z,Z,Z,Z)-3,6,9,12,15-pentacosapentaene.

3. A synthetic pheromone composition comprising at least one straight-chain pentaene having at least about 19 carbon atoms in the chain.

4. A synthetic pheromone of claim 3, wherein the straight-chain pentaene having at least about 19 carbon atoms is selected from the group consisting of 3,6,9,12,15-tricosapentaene and 3,6,9,12,15-pentacosapentaene.

5. The synthetic pheromone composition of claim 4, comprising (Z,Z,Z,Z,Z)-3,6,9,12,15-tricosapentaene and (Z,Z,Z,Z,Z)-3,6,9,12,15-pentacosapentaene.

6. The synthetic pheromone composition of claim 4, further comprising 11,13-hexadecadienal, ethyl palmitate and ethyl-11,13-hexadecadienoate.

7. The synthetic pheromone composition of claim 6, comprising (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate and ethyl (Z,Z)-11,13-hexadecadienoate.

8. The synthetic pheromone composition of claim 6, comprising 3,6,9,12,15-tricosapentaene, 3,6,9,12,15-pentacosapentaene, 11,13-hexadecadienal, ethyl palmitate, ethyl-11,13-hexadecadienoate and 11,13-hexadecadien-1-yl acetate.

9. The synthetic pheromone composition of claim 8, comprising (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate, ethyl (Z,Z)-11,13-hexadecadienoate and (Z,Z)-11,13-hexadecadien-1-yl acetate.

10. The synthetic pheromone composition of claim 9, comprising compounds in about the following ratio: (Z,Z,Z,Z,Z)-3,6,9,12,15-tricosapentaene, 15; (Z,Z,Z,Z,Z)-3,6,9,12,15-pentacosapentaene, 17; (Z,Z)-11,13-hexadecadienal, 100; ethyl palmitate, 14; ethyl (Z,Z)-11,13-hexadecadienoate, 5; and (Z,Z)-11,13-hexadecadien-1-yl acetate, 5.

11. An insect pest trap comprising a trap and a synthetic pheromone composition comprising at least one straight-chain pentaene having at least about 19 carbon atoms in the chain.

12. The insect pest trap of claim 11, wherein the synthetic pheromone composition comprises 3,6,9,12,15-tricosapentaene and 3,6,9,12,15-pentacosapentaene.

13. The insect pest trap of claim 12, wherein the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate and ethyl (Z,Z)-11,13-hexadecadienoate.

14. The insect pest trap of claim 13, wherein the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate, ethyl (Z,Z)-11,13-hexadecadienoate and (Z,Z)-11,13-hexadecadien-1-yl acetate.

15. A method for attracting an insect pest using an insect pest trap comprising a trap and a synthetic pheromone composition comprising 3,6,9,12,15-tricosapentaene and 3,6,9,12,15-pentacosapentaene.

16. The method of claim 15, wherein the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate and ethyl (Z,Z)-11,13-hexadecadienoate.

17. The method of claim 15, wherein the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate, ethyl (Z,Z)-11,13-hexadecadienoate and (Z,Z)-11,13-hexadecadien-1-yl acetate.

18. The method of claim 15, wherein the insect pest is a navel orangeworm, Amyelois transitella Walker.

19. The method of claim 15, wherein the subfamily is Pyralinae.

20. The method of claim 19, wherein the insect pest is a meal moth, Pyralis farinalis Linnaeus.

21. A method for inhibiting an insect pest using a synthetic pheromone composition comprising (Z,Z)-11,13-hexadecadien-1-yl acetate.

22. The method of claim 21, wherein the insect pest is a meal moth, Pyralis farinalis Linnaeus.

23. A method for disrupting mating of an insect pest, the method comprising releasing a synthetic pheromone composition comprising 3,6,9,12,15-tricosapentaene and 3,6,9,12,15-pentacosapentaene in a amount sufficient to disrupt mating of the insect pest.

24. The method of claim 23, wherein the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate and ethyl (Z,Z)-11,13-hexadecadienoate.

25. The method of claim 23, wherein the synthetic pheromone composition comprises (Z,Z,Z,Z,Z)-3,6,9,12,15-tricosapentaene, (Z,Z,Z,Z,Z)-3,6,9,12,15-pentacosapentaene, (Z,Z)-11,13-hexadecadienal, ethyl palmitate, ethyl (Z,Z)-11,13-hexadecadienoate and (Z,Z)-11,13-hexadecadien-1-yl acetate.
